(19) United States
(12) Patent Application Publication
(10) Pub. No.: US 2018/0049912 A1
(43) Pub. Date: Feb. 22, 2018

(54) PHYSIOLOGICAL ADJUSTMENT SYSTEM, INTELLIGENT UNDERPANTS AND PHYSIOLOGICAL ADJUSTMENT METHOD

(71) Applicants: BOE Technology Group Co., Ltd., Beijing (CN); BOE Optical Science Technology Co., Ltd., Suzhou (CN)

(72) Inventor: Ling ZHU, Beijing (CN)

(21) Appl. No.: 15/543,646

(22) PCT Filed: May 11, 2016
(86) PCT No.: PCT/CN2016/081670

§ 371 Date: Jul. 14, 2017

(30) Foreign Application Priority Data
Mar. 21, 2016 (CN) 201610161082.7

(51) Int. Cl. A61F 7/02; A41B 9/04; A61B 10/00; A61B 5/145; A61B 5/00

(52) U.S. Cl. CPC A61F 7/02; A41B 9/04; A61B 10/0012; A61B 5/14546; A61F 2007/0022; A61B 5/4836; A61B 5/6804; A61B 5/0024; A61B 10/007

(57) ABSTRACT

A physiological adjustment system, intelligent underpants including the physiological adjustment system and a physiological adjustment method for implementing a physiological adjustment using the physiological adjustment system are disclosed. The physiological adjustment system includes: a heating module for heating a human body based on a received heating command; a detection module for detecting a physiological state to obtain physiological state information; and a control module for sending the heating command to the heating module. The heating command includes a first heating command generated based on the physiological state information.

### CROSS-REFERENCE TO RELATED APPLICATION

This application is a Section 371 National Stage Application of International Application No. PCT/CN2016/081670, filed on May 11, 2016, entitled “PHYSIOLOGICAL ADJUSTMENT SYSTEM, INTELLIGENT UNDERPANTS AND PHYSIOLOGICAL ADJUSTMENT METHOD”, which claims priority to Chinese Patent Application No. 201610161082.7, filed on Mar. 21, 2016 with SIPO, incorporated herein by reference in their entirety.

### BACKGROUND

### Technical Field

Embodiments of the present disclosure relate to a field of physiological adjustment, and more particularly, to a physiological adjustment system, intelligent underpants and a physiological adjustment method.

### Description of the Related Art

Most of existing underclothes only have an ordinary function, i.e., functioning as clothes, but cannot adjust physiology of a human body according to physiological state information of the human body. Especially for women, in the early menstrual period and during the whole menstrual period, some symptoms such as cold uterus, dysmenorrhea and the like occur due to a low uterus temperature, which makes a majority of women miserable and even seriously affects daily work and daily life of the women.

Therefore, it is very urgent to provide a device and a method capable of monitoring the physiological state information of the human body and adjusting the physiology of the human body.

### SUMMARY

According to an embodiment of the present disclosure, there is provided a physiological adjustment system, comprising: a heating module for heating a human body based on a received heating command; a detection module for detecting a physiological state to obtain physiological state information; and a control module for sending the heating command to the heating module, wherein the heating command comprises a first heating command generated based on the physiological state information.

In a non-limiting embodiment, the physiological state information comprises first physiological state information, and the control module is further configured for determining whether to generate the first heating command based on the first physiological state information.

In a non-limiting embodiment, the first physiological state information comprises state information regarding whether a woman is in a menstrual period, and the control module is further configured for determining whether to generate the first heating command based on the state information regarding whether the woman is in the menstrual period.

In a non-limiting embodiment, the physiological state information comprises second physiological state information different from the first physiological state information, the first heating command is generated based on the second physiological state information, and the control module is further configured for generating a physiological result based on the second physiological state information and determining whether to generate the first heating command based on the physiological result.

In a non-limiting embodiment, the second physiological state information comprises state information regarding whether a woman is in an ovulation period, the physiological result comprises woman menstrual period information, and the control module is further configured for generating the woman menstrual period information based on the state information regarding whether the woman is in the ovulation period and determining whether to generate the first heating command based on the woman menstrual period information.

In a non-limiting embodiment, the physiological adjustment system further comprises a communication module for receiving a command from an external decision device and transmitting the physiological state information detected by the detection module to the external decision device, wherein the external decision device is configured for generating a start command based on the transmitted physiological state information, and the control module is configured for receiving the start command and determining whether to generate a second heating command based on the start command.

In a non-limiting embodiment, the control module is configured for generating only one of the first heating command and the second heating command in a preset period of time.

In a non-limiting embodiment, the external decision device comprises an intelligent terminal comprising an application program for generating the start command based on the transmitted physiological state information.

In a non-limiting embodiment, the communication module comprises a wireless communication module.

In a non-limiting embodiment, the physiological state information transmitted by the communication module comprises state information regarding whether a woman is in a menstrual period and/or state information regarding whether a woman is in an ovulation period, and

the control module is configured for determining whether to generate the start command based on the state information regarding whether the woman is in the menstrual period; and/or

the control module is configured for generating woman menstrual period information based on the state information regarding whether the woman is in the ovulation period and for determining whether to generate the start command based on the woman menstrual period information.

In a non-limiting embodiment, the physiological adjustment system further comprises a flexible display capable of displaying at least one kind of information from an information group consisting of information regarding whether a woman is in a menstrual period, a body temperature, and a mood sign indicating physiological state information of the woman and information regarding whether the woman is in an ovulation period.

In a non-limiting embodiment, the at least one kind of information from the information group is transmitted to an external intelligent terminal by the communication module.

In a non-limiting embodiment, the control module comprises a manual heating switch, and the heating command further comprises a third heating command generated by the manual heating switch.

According to another embodiment of the present disclosure, there are provided intelligent underpants, comprising the physiological adjustment system according to the above embodiments, wherein the heating module is arranged at a position corresponding to a lower abdomen of a wearer on a front side of the underpants.

In a non-limiting embodiment, the intelligent underpants further comprise an insulating bag filled with essential oil, and a heat generation source of the heating module is provided in the essential oil.

In a non-limiting embodiment, the detection module comprises a blood sensor for detecting blood to obtain state information regarding whether a woman is in a menstrual period.

In a non-limiting embodiment, the detection module comprises an electronic ovulation test strip sensor provided in a crotch portion of the intelligent underpants, and the electronic ovulation test strip sensor is configured for detecting a peak level of luteinizing hormone in residual urine to obtain state information regarding whether a woman is in an ovulation period.

In a non-limiting embodiment, the physiological adjustment system further comprises a temperature sensor for measuring an abdomen temperature of the wearer.

According to a further another embodiment of the present disclosure, there is provided a physiological adjustment method, comprising steps of:

detecting a physiological state to obtain physiological state information;

generating a heating command; and

heating a human body based on the heating command,

wherein the heating command comprises a first heating command generated based on the physiological state information.

In a non-limiting embodiment, the physiological state information comprises first physiological state information, and the method further comprises determining whether to generate the first heating command based on the first physiological state information.

In a non-limiting embodiment, the first physiological state information comprises state information regarding whether a woman is in a menstrual period, and it is determined whether to generate the first heating command based on the state information regarding whether the woman is in the menstrual period.

In a non-limiting embodiment, the physiological state information comprises second physiological state information different from the first physiological state information, the first heating command is generated based on the second physiological state information, and the method further comprises generating a physiological result based on the second physiological state information and determining whether to generate the first heating command based on the physiological result.

In a non-limiting embodiment, the second physiological state information comprises state information regarding whether a woman is in an ovulation period, the physiological result comprises woman menstrual period information, and the woman menstrual period information is generated based on the state information regarding whether the woman is in the ovulation period and it is determined whether to generate the first heating command based on the woman menstrual period information.

In a non-limiting embodiment, the method further comprises steps of:

transmitting the detected physiological state information to an external decision device;

generating a start command based on the transmitted physiological state information; and

receiving the start command and determining whether to generate a second heating command based on the start command.

In a non-limiting embodiment, the step of determining whether to generate the second heating command based on the start command comprises:

generating only one of the first heating command and the second heating command in a preset period of time.

In a non-limiting embodiment, the transmitted physiological state information comprises state information regarding whether a woman is in a menstrual period and/or state information regarding whether a woman is in an ovulation period, and

the step of generating the start command based on the transmitted physiological state information comprises:

determining whether to generate the start command based on the state information regarding whether the woman is in the menstrual period; and/or

generating woman menstrual period information based on the state information regarding whether the woman is in the ovulation period and determining whether to generate the start command based on the woman menstrual period information.

In a non-limiting embodiment, the heating command further comprises a third heating command generated manually.

### BRIEF DESCRIPTION OF THE DRAWINGS

FIG. 1 is a schematic functional block diagram of a physiological adjustment system according to an embodiment of the present disclosure;

FIG. 2 is a schematic circuit diagram of a physiological adjustment system according to an embodiment of the present disclosure;

FIG. 3 shows intelligent underpants according to an embodiment of the present disclosure; and

FIG. 4 shows a physiological adjustment method according to an embodiment of the present disclosure.

### DETAILED DESCRIPTION OF PREFERRED EMBODIMENTS

The embodiments of the present disclosure will now be described in detail with reference to the accompanying drawings, in which similar reference numerals refer to similar parts.

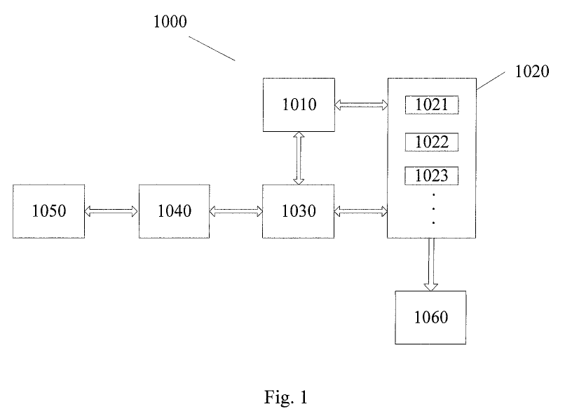

FIG. 1 is a schematic functional block diagram of a physiological adjustment system 1000 according to the present disclosure. The physiological adjustment system 1000 comprises: a heating module 1010 for heating a human body based on a received heating command; a detection module 1020 for detecting a physiological state to obtain physiological state information; and a control module 1030 for sending the heating command to the heating module 1010, wherein the heating command comprises a first heating command generated based on the physiological state information. The physiological adjustment system according to the present disclosure can heat the body based on the physiological state information of a user, so as to alleviate pain caused by dysmenorrhea, cold uterus and the like of the user.

In an embodiment of the present disclosure, the physiological state information comprises first physiological state information, and the control module is further configured for determining whether to generate the first heating command based on the first physiological state information. The physiological state information comprises state information regarding whether a woman is in a menstrual period, and the control module is further configured for determining whether to generate the first heating command based on the state information regarding whether the woman is in the menstrual period.

In an embodiment of the present disclosure, the detection module 1020 may be arranged on clothes (for example, underpants) and comprise at least one sensor for measuring a physiological index of a human body. For example, the detection module 1020 may comprise a blood sensor 1021 capable of detecting blood, and the detection module 1020 may obtain state information regarding whether a woman is in a menstrual period through the detection from the blood sensor 1021. The physiological state information comprises state information regarding whether a woman is in a menstrual period, and the control module 1030 is further configured for determining whether to generate the first heating command based on the state information regarding whether the woman is in the menstrual period. It should be noted that the first physiological state information may also comprise other physiological state information as long as it can reflect physiological characteristics of the human body and human discomfort may be alleviated by heating means based on the physiological characteristics.

In an embodiment of the present disclosure, the physiological state information comprises second physiological state information different from the first physiological state information, the first heating command is generated based on the second physiological state information, and the control module is further configured for generating a physiological result based on the second physiological state information and determining whether to generate the first heating command based on the physiological result.

In an embodiment of the present disclosure, the second physiological state information comprises state information regarding whether a woman is in an ovulation period, the physiological result comprises woman menstrual period information, and the control module is further configured for generating the woman menstrual period information based on the state information regarding whether the woman is in an ovulation period and for determining whether to generate the first heating command based on the woman menstrual period information.

In an embodiment of the present disclosure, the detection module 1020 may further comprise an electronic ovulation test strip sensor 1022 capable of detecting a peak level of luteinizing hormone (LH) in residual urine on an underwear. For example, the luteinizing hormone (LH) in the urine appears at a high peak in the first 24 to 48 hours of the ovulation period of the woman, then the detection module 1020 may obtain the state information regarding whether the woman is in an ovulation period based on a detection result from the electronic ovulation test strip sensor 1022.

In an embodiment of the present disclosure, the detection module 1020 further comprises a temperature sensor 1023 capable of measuring a body temperature of the user, so that the user may timely know the body temperature of himself. It will be appreciated by those skilled in the art that the above-described sensors are merely exemplary sensors, but are not intended to limit the present disclosure. The detection module 1020 is not limited to the inclusion of the above-described sensors, but it may include other sensors for measuring specific parameters if required.

In an embodiment of the present disclosure, the control module 1030 may determine whether to generate the first heating command based on the first state information regarding whether the woman is in the menstrual period. In addition, the control module 1030 may generate a physiological result based on the second physiological state information regarding whether the woman is in an ovulation period, the physiological result comprises woman menstrual period information, and the control module 1030 is further configured for determining whether to generate the first heating command based on the woman menstrual period information. Specifically, the menstrual period information includes cycle information of the menstrual period. For example, after obtaining the state information that the woman is in the ovulation period, a starting date of the menstrual period may be determined by adding the ovulation period by about one week, then the cycle of the menstrual period of the woman is a period of 5 to 7 days after the starting date of the menstrual period. In an embodiment of the present disclosure, the control module 1030 determines whether to generate a start command during the menstrual cycle, for example, the start command may be sent out during the menstrual cycle only one time, or the start command may be sent out during the menstrual cycle at intervals. The control module 1030 may not generate any heating command at the end of the cycle of the menstrual period so as to satisfy different individual requirements.

In addition, the physiological adjustment system 100 further comprises a communication module 1040 for receiving a command from an external decision device 1050 and transmitting the physiological state information detected by the detection module 1020 to the external decision device 1050. The transmitted physiological state information comprises state information regarding whether a woman is in a menstrual period and/or state information regarding whether a woman is in an ovulation period, a body temperature of the user, a mood sign indicating physiological state information of the woman and the like. The mood sign may include an unhappy face (for example, a rainy expression) that indicates a woman is in a menstrual period, a face (for example, a smiling face) that indicates the woman is in a safe period, and a face (for example, a small hand) that indicates the woman is in an ovulation period.

The external decision device 1050 may generate a start command based on the transmitted physiological state information. Specifically, the external decision device 1050 determines whether to generate the start command based on the state information regarding whether the woman is in the menstrual period. Optionally, when the woman is in the menstrual period, the start command is generated. Alternatively, the external decision device 1050 generates the woman menstrual period information based on the state information regarding whether the woman is in the ovulation period and determines whether to generate the start command based on the woman menstrual period information. For example, after obtaining the state information that the woman is in the ovulation period, a starting date of the menstrual period may be determined by adding the ovulation period by about one week, then the cycle of the menstrual period of the woman is a period of 5 to 7 days after the starting date of the menstrual period, the external decision device 1050 determines whether to generate the start command during the menstrual cycle, for example, the start command may be sent out during the menstrual cycle only one time, or the start command may be sent out during the menstrual cycle at intervals. The control module 1030 receives the start command and determines whether to generate a second heating command based on the start command. Determining whether to generate the second heating command based on the start command includes generating only one of the first heating command and the second heating command in a preset period of time. In this way, it is possible to prevent the first heating command and the second heating command from repeating, thereby optimizing the system. The external decision device 1050 comprises an intelligent terminal comprising an application program for generating the start command based on the transmitted physiological state information. In this embodiment, the external decision device 1050 may be a smartphone in which an application program for controlling the heating module is installed. The communication module 1040 comprises a wireless communication module, such that it is wirelessly connected to the external decision device 1050, for example via a Bluetooth technology, a WLAN network, an infrared communication technology or the like. The external decision device 1050 may also record and calculate the menstrual cycle of the woman using the physiological state information transmitted by the communication module 1040, and transmit the heating command to the heating module 1010 during the menstrual cycle.

The physiological adjustment system 1000 further comprises a flexible display 1060 connected to the control module 1030 and capable of visually displaying the physiological state information for the user, for example, the body temperature of the user, information regarding whether a woman is in a menstrual period, a mood sign indicating physiological state of the woman, information regarding whether a woman is in an ovulation period and the like. The mood sign may include an unhappy face (for example, a rainy expression) that indicates a woman is in a menstrual period, a face (for example, a smiling face) that indicates the woman is in a safe period, and a face (for example, a small hand) that indicates the woman is in an ovulation period. Of course, the mood sign is not limited to the listed examples, but may include any possible face signs that reflect the physiological state.

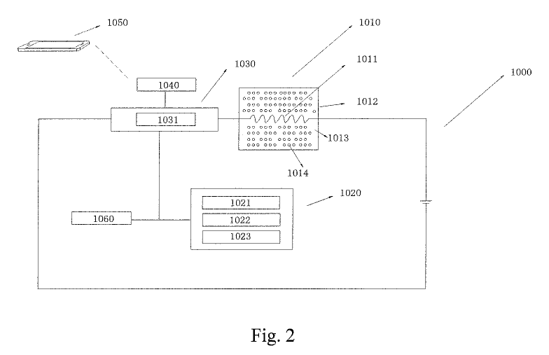

According to an embodiment of the present disclosure, the control module 1030 may further comprise a manual heating switch 1031 (referring to FIG. 2), and the heating command further comprises a third heating command generated by the manual heating switch 1031.

FIG. 2 is a schematic circuit diagram of a physiological adjustment system 1000 according to an embodiment of the present disclosure, in which the principle of a heating circuit is shown predominantly.

In an embodiment of the present disclosure, the heating module 1010 includes an iron-chromium-aluminum alloy heat pipe 1011 functioning as a heating element and a flexible bag container 1012. The iron-chromium-aluminum alloy heat pipe 1011 is arranged in the flexible bag container 1012 filled with liquid essential oil 1013. In an embodiment of the present disclosure, a massage stone 1014 is also provided in the flexible bag container 1012. By means of the essential oil 1013 and the massage stone 1014, it can achieve a uniform heating and massage effect. It should be understood that the essential oil 1013 and the massage stone 1014 themselves may be made from insulation materials, and the flexible bag container 1012 may also be made of an insulation material. When the physiological adjustment system 1000 is applied to a woman's underpants, it is easily to be worn and can alleviate the dysmenorrhea symptom of the woman.

The blood sensor 1021, the electronic ovulation test strip sensor 1022 and the temperature sensor 1023 can respectively detect the blood, the peak level of the luteinizing hormone (LH) in the residual urine on the underwear and the human body temperature, so that it can obtain the physiological state information regarding whether the woman is in the menstrual period and whether the woman is in the ovulation period and the body temperature of the woman. The flexible display 1060 is configured for visually displaying the physiological state information for the user, such as, the body temperature of the user, information regarding whether a woman is in a menstrual period, a mood sign indicating physiological state of the woman, information regarding whether a woman is in an ovulation period and the like. The mood sign may include an unhappy face (for example, a rainy expression) that indicates a woman is in a menstrual period, a face (for example, a smiling face) that indicates the woman is in a safe period, and a face (for example, a small hand) that indicates the woman is in an ovulation period.

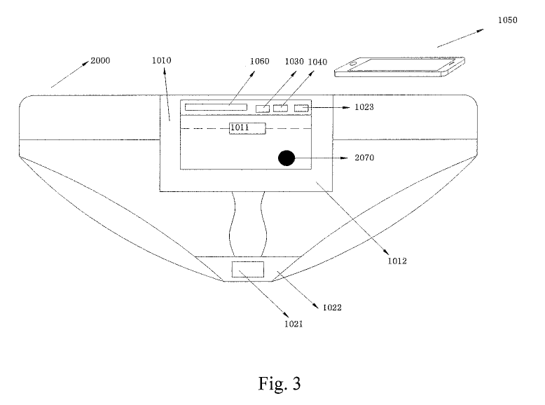

FIG. 3 shows intelligent underpants 2000 according to an embodiment of the present disclosure. The intelligent underpants 2000 comprises the above physiological adjustment system. The heating module 1010 is arranged at a position corresponding to a lower abdomen of a wearer on a front side of the underpants 2000. The heating module 1010 adopts an iron-chromium-aluminum alloy heat pipe 1011 to act as a heating element. The iron-chromium-aluminum alloy heat pipe 1011 is arranged in a flexible bag container 1012 filled with liquid essential oil and a massage stone.

The detection module 1020 includes a blood sensor 1021, an electronic ovulation test strip sensor 1022, and a temperature sensor 1023. The blood sensor 1021 is arranged at a crotch portion of the underpants 2000 close to the lower abdomen portion. The electronic ovulation test strip sensor 1022 is arranged at the crotch portion of the underpants and detects a peak level of luteinizing hormone in residual urine to further obtain the state information regarding whether the woman is in the ovulation period, and displays it by the flexible display 1060, thereby assisting the woman in preparing for pregnancy and increasing chance of pregnancy. The temperature sensor 1023 is capable of measuring the abdomen temperature of the wearer.

The communication module 1040 may be arranged at an upper side of the underpants 2000 close to a waist to facilitate communicating with the external smartphone 1050. It should be understood that the communication can be implemented as long as the communication module 1040 is arranged on the underpants 2000, therefore, the detailed arrangement position will not be limited in the embodiments of the present disclosure.

The flexible display 1060 is also arranged on the upper side of the underpants 2000 close to the waist on the front side, and configured for visually displaying the physiological state information for the user, such as, the body temperature of the user, information regarding whether a woman is in a menstrual period, a mood sign indicating physiological state of the woman, information regarding whether a woman is in an ovulation period and the like. The mood sign may include an unhappy face (for example, a rainy expression) that indicates a woman is in a menstrual period, a face (for example, a smiling face) that indicates the woman is in a safe period, and a face (for example, a small hand) that indicates the woman is in an ovulation period.

The control module 1030 and its manual heating switch 1031 are provided on the front side of the underpants 2000 for ease of operation.

The intelligent underpants 2000 can also be provided with an indicator light 2070. The indicator light 2070 may be a color-changeable LED lamp so as to indicate the relevant working status information of the underpants. For example, a flashing red light indicates lack of electricity, a flashing green light indicates that the underpants are in an heating status. It will be understood by those skilled in the art that the indicator light may work according to a presetting such that different lighting statuses indicate different working status information, and the above-described working status information is merely exemplary, and it is possible to set the working status information of the indicator light by those skilled in the art as required.

The intelligent underpants 2000 according to the present disclosure may be dry-cleaned or brushed by using detergent.

It will be appreciated by those skilled in the art that the positions of the various components of the intelligent underpants 2000 are exemplary, but not restrictive. The above-described components may be adjusted according to requirements or design preferences, without adversely affecting the implementation of their functionality.

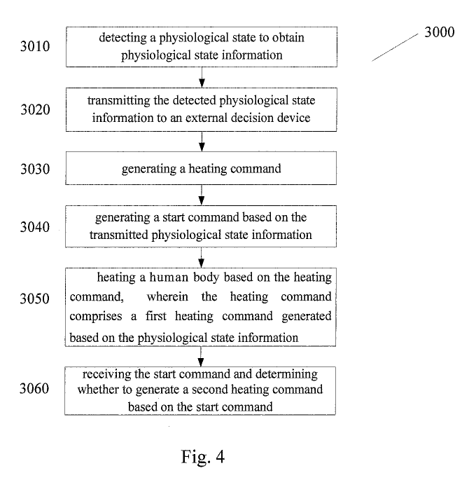

FIG. 4 shows a physiological adjustment method 3000 according to an embodiment of the present disclosure. The physiological adjustment method comprises following steps:

step 3010: detecting a physiological state to obtain physiological state information;

step 3030: generating a heating command; and

step 3050: heating a human body based on the heating command, wherein the heating command comprises a first heating command generated based on the physiological state information.

According to an embodiment of the present disclosure, the physiological state information comprises first physiological state information, and the method further comprises a step of determining whether to generate the first heating command based on the first physiological state information. The first physiological state information comprises state information regarding whether a woman is in a menstrual period, and it is determined whether to generate the first heating command based on the state information regarding whether the woman is in the menstrual period.

The physiological state information further comprises second physiological state information different from the first physiological state information, the first heating command is generated based on the second physiological state information, and the method further comprises a step of generating a physiological result based on the second physiological state information and determining whether to generate the first heating command based on the physiological result. The second physiological state information comprises state information regarding whether a woman is in an ovulation period, the physiological result comprises woman menstrual period information, and the woman menstrual period information is generated based on the state information regarding whether the woman is in the ovulation period and it is determined whether to generate the first heating command based on the woman menstrual period information.

The method further comprises following steps:

step 3020: transmitting the detected physiological state information to an external decision device;

step 3040: generating a start command based on the transmitted physiological state information; and

step 3060: receiving the start command and determining whether to generate a second heating command based on the start command.

The step 3060 of determining whether to generate the second heating command based on the start command comprises: generating only one of the first heating command and the second heating command in a preset period of time. That is to say, the step 3010, the step 3030, the step 3050 and the steps 3020, 3040, 3060 may be performed in parallel, and have no interference with each other, and the step number does not mean the relevant steps must be performed in this order. The sequence of the steps in this embodiment is merely exemplary, but not restrictive, and the steps may be adjusted in the aspect of sequence according to the actual situation. However, only one of the first heating command and the second heating command is generated in a preset period of time, that is, only one heating command is generated in the preset period of time.

The transmitted physiological state information comprises state information regarding whether a woman is in a menstrual period and/or state information regarding whether a woman is in an ovulation period, and

the step of generating the start command based on the transmitted physiological state information comprises:

determining whether to generate the start command based on the state information regarding whether the woman is in the menstrual period; and/or

generating woman menstrual period information based on the state information regarding whether the woman is in the ovulation period and determining whether to generate the start command based on the woman menstrual period information. The menstrual period information includes cycle information of the menstrual period.

The heating command further comprises a third heating command generated manually.

The physiological adjustment system and the physiological adjustment method according to embodiments of the present disclosure can detect physiological state of a user, and heat the body based on the physiological state information of the user, so as to alleviate pain caused by dysmenorrhea, cold uterus and the like of the user. Additionally, the intelligent underpants according to embodiments of the present disclosure act as an intelligent wearable device, improve technological element of the clothes, adjust physiological parameters of the human body by scientific and technological means, and increase additional values of product.

The present disclosure has been described in detail with reference to the embodiments above. It will be understood by those skilled in the art that the above-described embodiments are merely exemplary, but not to limit the present disclosure. Variations and modifications as well as equivalent alternatives may be made by those skilled in the art without departing from the scope of the present disclosure defined in the claims. Those technical features mentioned in the present disclosure can be freely combined to form various technical solutions without conflict.

1. A physiological adjustment system, comprising:
a heating module for heating a human body based on a received heating command;
a detection module for detecting a physiological state to obtain physiological state information; and
a control module for sending the heating command to the heating module, wherein the heating command comprises a first heating command generated based on the physiological state information.

2. The physiological adjustment system according to claim 1, wherein the physiological state information comprises first physiological state information, and the control module is further configured for determining whether to generate the first heating command based on the first physiological state information.

3. The physiological adjustment system according to claim 2, wherein the first physiological state information comprises state information regarding whether a woman is in a menstrual period, and the control module is further configured for determining whether to generate the first heating command based on the state information regarding whether the woman is in the menstrual period.

4. The physiological adjustment system according to claim 3, wherein the physiological state information comprises second physiological state information different from the first physiological state information, the first heating command is generated based on the second physiological state information, and the control module is further configured for generating a physiological result based on the second physiological state information and determining whether to generate the first heating command based on the physiological result.

5. The physiological adjustment system according to claim 4, wherein the second physiological state information comprises state information regarding whether a woman is in an ovulation period, the physiological result comprises woman menstrual period information, and the control module is further configured for generating the woman menstrual period information based on the state information regarding whether the woman is in the ovulation period and determining whether to generate the first heating command based on the woman menstrual period information.

6. The physiological adjustment system according to claim 1, further comprising a communication module for receiving a command from an external decision device and transmitting the physiological state information detected by the detection module to the external decision device, wherein the external decision device is configured for generating a start command based on the transmitted physiological state information, and the control module is configured for receiving the start command and determining whether to generate a second heating command based on the start command.

7. (canceled)

8. (canceled)

9. (canceled)

10. (canceled)

11. The physiological adjustment system according to claim 6, further comprising a flexible display capable of displaying at least one kind of information from an information group consisting of information regarding whether a woman is in a menstrual period, a body temperature, and a mood sign indicating physiological state information of the woman and information regarding whether the woman is in an ovulation period.

12. (canceled)

13. (canceled)

14. Intelligent underpants, comprising the physiological adjustment system according to claim 1, wherein the heating module is arranged at a position corresponding to a lower abdomen of a wearer on a front side of the underpants.

15. The intelligent underpants according to claim 14, further comprising an insulating bag filled with essential oil, and a heat generation source of the heating module is provided in the essential oil.

16. The intelligent underpants according to claim 14, wherein the detection module comprises a blood sensor for detecting blood to obtain state information regarding whether a woman is in a menstrual period.

17. The intelligent underpants according to claim 14, wherein the detection module comprises an electronic ovulation test strip sensor provided in a crotch portion of the intelligent underpants, and the electronic ovulation test strip sensor is configured for detecting a peak level of luteinizing hormone in residual urine to obtain state information regarding whether a woman is in an ovulation period.

18. The intelligent underpants according to claim 14, wherein the physiological adjustment system further comprises a temperature sensor for measuring an abdomen temperature of the wearer.

19. A physiological adjustment method, comprising steps of:
detecting a physiological state to obtain physiological state information;
generating a heating command; and
heating a human body based on the heating command,
wherein the heating command comprises a first heating command generated based on the physiological state information.

20. The physiological adjustment method according to claim 19, wherein the physiological state information comprises first physiological state information, and the method further comprises determining whether to generate the first heating command based on the first physiological state information.

21. The physiological adjustment method according to claim 20, wherein the first physiological state information comprises state information regarding whether a woman is in a menstrual period, and it is determined whether to generate the first heating command based on the state information regarding whether the woman is in the menstrual period.

22. The physiological adjustment method according to claim 21, wherein the physiological state information comprises second physiological state information different from the first physiological state information, the first heating command is generated based on the second physiological state information, and the method further comprises generating a physiological result based on the second physiological state information and determining whether to generate the first heating command based on the physiological result.

23. The physiological adjustment method according to claim 22, wherein the second physiological state information comprises state information regarding whether a woman is in an ovulation period, the physiological result comprises woman menstrual period information, and the woman menstrual period information is generated based on the state information regarding whether the woman is in the ovulation period and it is determined whether to generate the first heating command based on the woman menstrual period information.

24. The physiological adjustment method according to claim 19, wherein the method further comprises steps of:
transmitting the detected physiological state information to an external decision device;
generating a start command based on the transmitted physiological state information; and
receiving the start command and determining whether to generate a second heating command based on the start command.

25. The physiological adjustment method according to claim 24, wherein the step of determining whether to generate the second heating command based on the start command comprises:
generating only one of the first heating command and the second heating command in a preset period of time.

26. The physiological adjustment method according to claim 24, wherein the transmitted physiological state information comprises state information regarding whether a woman is in a menstrual period and/or state information regarding whether a woman is in an ovulation period, and
the step of generating the start command based on the transmitted physiological state information comprises:
determining whether to generate the start command based on the state information regarding whether the woman is in the menstrual period; and/or
generating woman menstrual period information based on the state information regarding whether the woman is in the ovulation period and determining whether to generate the start command based on the woman menstrual period information.

27. (canceled)
